SEQUENCE LISTING (14 sequences)

SEQ ID NO: 1 - DNA, 3084 bp, human
  CDS (655)..(1854)
aagatctaaaaacggacatctccaccgtgggtggctcctttttctttttctttttttcccacccttcaggaagtggacgtttcgttatcttctgatccttgcaccttcttttggggaaacggggcccttctgcccagatcccctctcttttctcggaaaacaaactactaagtcggcatccggggtaactacagtggagagggtttccgcggagacgcgccgccggaccctcctctgcactttggggaggcgtgctccctccagaaccggcgttctccgcgcgcaaatcccggcgacgcggggtcgcggggtggccgccggggcagcctcgtctagcgcgcgccgcgcagacgcccccggagtcgccagctaccgcagccctcgccgcccagtgcccttcggcctcggggcgggcgcctgcgtcggtctccgcgaagcgggaaagcgcggcggccgccgggattcgggcgccgcggcagctgctccggctgccggccggcggccccgcgctcgcccgccccgcttccgcccgctgtcctgctgcacgaacccttccaactctcctttcctcccccacccttgagttacccctctgtctttcctgctgttgcgcgggtgctcccacagcggagcggagattacagagccgccgggatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctgaaacatggtggaaaacgaagctcattcccaacgtgcaaagccaaggcagcgaccccaggacctcttctggagatggaagcttgttgaaaacccagactgtctccacggcctgcccagtcgacgccaaaggaacactgacatcaattttaccctgaggtcactgctagagacgctgatccataaagacaatcactgccaacccctctttcgtctactgcaagagccaagttccaaaataaagcataaaaaggttttttaaaaggaaatgtaaaagcacatgagaatgctagcaggctgtggggcagctgagcagcttttctccccccatatctgcgtgcacttcccagagcatcttgcatccaaacctgtaacctttcggcaaggacggtaacttggctgcatttgcctgtcatgcgcaactggagccagcaaccagctatccatcagcaccccagtggaggagttcatggaagagttccctctttgtttctgcttcatttttctttcttttcttttctcctaaagcttttatttaacagtgcaaaaggatcgtttttttttgcttttttaaacttgaatttttttaatttacactttttagttttaattttcttgtatattttgctagctatgagcttttaaataaaattgaaagttctggaaaagtttgaaataatgacataaaaagaagccttctttttctgagacagcttgtctggtaagtggcttctctgtgaattgcctgtaacacatagtggcttctccgcccttgtaaggtgttcagtagagctaaataaatgtaatagccaaaccccactctgttggtagcaattggcagccctatttcagtttattttttcttctgttttcttcttttctttttttaaacagtaaaccttaacagatgcgttcagcagactggtttgcagtgaattttcatttctttccttatcacccccttgttgtaaaaagcccagcacttgaattgttattactttaaatgttctgtatttgtatctgtttttattagccaattagtgggattttatgccagttgttaaaatgagcattgatgtacccattttttaaaaaagcaagcacagcctttgcccaaaactgtcatcctaacgtttgtcattccagtttgagttaatgtgctgagcatttttttaaaagaagctttgtaataaaacatttttaaaaattgtcatttaaaaaaaaaaaaaaaaaa

SEQ ID NO: 2 - protein, 399 aa, human
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPYMSNGSLSPPIPRTSNKVPVVQPSHAVHPLTPLITYSDEHFSPGSHPSHIPSDVNSKQGMSRHPPAPDIPTFYPLSPGGVGQITPPLGWQGQPVYPITGGFRQPYPSSLSVDTSMSRFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESASGTGPRMTAAYI

SEQ ID NO: 3 - DNA, 18 bp, artificial
  primer forward LEF-1
cttggctggcaaggtcag

SEQ ID NO: 4 - DNA, 19 bp, artificial
  primer reverse LEF-1 PCR
gagggatgccagttgtgtg

SEQ ID NO: 5 - DNA, 19 bp, artificial
  primer LEF-1 forward
ccgaagaggaaggcgttta

SEQ ID NO: 6 - DNA, 18 bp, artificial
  primer LEF-1 reverse
gctggccgggatgatttc

SEQ ID NO: 7 - DNA, 64 bp, artificial
  shRNA LEF-1 forward
gatcccagtgcagctatcaaccagattcaagagatctggttgatagctgcactttttttggaag

SEQ ID NO: 8 - DNA, 65 bp, artificial
  shRNA probe reverse
tcgacttccaaaaaaaagtgcagctatcaaccagatctcttgaatctggttgatagctgcactgg

SEQ ID NO: 9 - DNA, 21 bp, artificial
  primer wt Noschift assy
gttctggctttgaaagagaat

SEQ ID NO: 10 - DNA, 21 bp, artificial
  noschift primer wt
caagaccgaaactttctctta

SEQ ID NO: 11 - DNA, 21 bp, artificial
  noshift primer mut 1
gttctggctccgaaagagaat

SEQ ID NO: 12 - DNA, 21 bp, artificial
  noshift primer mut 2
caagaccgaggctttctctta

SEQ ID NO: 13 - DNA, 23 bp, artificial
  primer competitor primer 1
aggagagaagcaaaggacactgc

SEQ ID NO: 14 - DNA, 23 bp, artificial
  nonspecific competitor 2
tcctctcttcgtttcctgtgacg

(19) United States
(12) Patent Application Publication
(10) Pub. No.: US 2010/0113343 A1
(43) Pub. Date: May 6, 2010

(54) METHODS INVOLVING LEF-1 REGULATION AND USE OF LEF-1 OR COMPOUNDS ALTERING LEF-1 SIGNALLING FOR TREATING OR PREVENTING DISEASES

(76) Inventors: Julia Skokowa, Hannover (DE); Karl Welte, Hannover (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/442,243

(22) PCT Filed: Sep. 24, 2007
(86) PCT No.: PCT/EP2007/008275

§ 371 Date: Oct. 26, 2009

Related U.S. Application Data
(60) Provisional application No. 60/846,342, filed on Sep. 22, 2006.

(51) Int. Cl. A61K 38/17; C07K 14/435; C07H 21/04; A61K 31/7088; C07H 21/02; C12Q 1/68; C12Q 1/02; C12N 5/02

(52) U.S. Cl. 514/12; 530/350; 536/23.5; 514/44 R; 536/24.5; 514/44 A; 435/6; 435/29; 435/375

(57) ABSTRACT

The present inventions relates to the use of LEF-1 or functional fragments or homologs thereof, or enhancer or inducer of LEF-1 expression, activity or LEF-1 mediated signalling for the preparation of a pharmaceutical for preventing or treating all types of cytopenia of the myeloid or lymphoid lineage. In particular, the present invention relates to the treatment of severe congenital neutropenia. In another embodiment the present invention relates to the treatment of various types of cancer, in particular, of cancer involving altered granulocyte proliferation, survival and differentiation from granulocytes progenitor cells.

The present inventions relates to the use of LEF-1 or functional fragments or homologs thereof, or enhancer or inducer of LEF-1 expression, activity or LEF-1 mediated signalling for the preparation of a pharmaceutical for preventing or treating all types of cytopenia of the myeloid or lymphoid lineage. In particular, the present invention relates to the treatment of severe congenital neutropenia. In another embodiment the present invention relates to the treatment of various types of cancer, in particular, of cancer involving altered granulocyte proliferation, survival and differentiation from granulocytes progenitor cells.

Further, the present invention concerns pharmaceutical compositions comprising as active ingredients both LEF-1 and G-CSF. Finally, the present inventions concern the treatment of cancer, in particular of AML or ALL inhibiting LEF-1 expression or signalling.

### PRIOR ART

Bone marrow failure syndromes are characterized by a deficiency of one or more hematopoietic lineage. A common feature of both congenital and acquired forms of bone marrow failure is a marked propensity to develop various types of leukemia, for example, acute myeloid leukemia (AML), acute lymphoid leukemia (ALL) or myelodysplastic syndrome (MDS). The myelodysplastic syndrome (MDS), also as known as preleukemia, represents a diverse collection of hematological conditions united by ineffective production of blood cells and varying risks of transformation to acute myelogenous leukemia. The anomalies include neutropenia and thrombocytopenia, abnormal granules in cells, abnormal nucleoshape and size etc. It is sought that MDS arises from mutations in the multi-potent bone marrow of stem cells, but the specific defects responsible for these diseases is remained poorly understood. As indicated above, various types of cytopenia including leukopenia may occur in MDS patients. The two most serious complications in MDS patients resulting from their cytopenia are bleeding or infection.

Further, various diseases are connected with or result from a differentiation blockage at various immature stages of differentiation, e.g. at different stages of myelopoiesis. Typical diseases associated with differentiation blockage are neutropenia characterized in a blockage of the differentiation into mature granulocytes, in particular, neutrophils. For example, severe congenital neutropenia (SCN) is a congenital bone marrow failure syndrome characterized by severe neutropenia present from birth, an arrest of myeloid differentiation at the promyelocyte/myelocyte stage and frequent infections. SCN is a rare disorder of the myelopoiesis characterized in a lack of peripheral blood neutrophils. Severe congenital neutropenia is considered to be a pre-leukemic syndrome, because more than 20 percent of patients with SCN progress to acute myelogenous leukemia (AML). All individuals with SCN have a characteristic bone marrow phenotype that distinguishes the condition from other neutropenias: “maturation arrest” with accumulation of granulocyte precursors (promyelocytes) and absence of mature granulocytes. The arrested SCN promyelocytes show impaired proliferation and differentiation in response to granulocyte colony-stimulating factor (G-CSF), as well as accelerated apoptosis. SCN is used as a model for investigating the regulation of myelopoiesis in humans due to its characteristic block in promyelocyte differentiation.

SCN has been described as a heterogeneous disorder involving mutations in various genes including those encoding neutrophil elastase (ELA2), HAX1, G-CSF receptor (G-CSFR), GFI-1, and WASP. Congenital neutropenia follows an autosomal dominant or autosomal recessive pattern of inheritance. In the beginning, bone marrow transplantation from ALL compatible donors was the only curative treatment option for congenital neutropenia patients. Both, the prognosis and the quality of life of congenital neutropenia patients, improved dramatically following the introduction of granulocytes colony-simulating factor therapy in 1987. More than 90% of congenital neutropenia patients responded to G-CSF treatment with an increase in the absolute neutrophil count (ANC). Hematopoietic stem cell transplantation remains the only currently available treatment for those patients refractory to G-CSF and patients were transformed into myelodysplastic syndrome. The diagnosis of SCN is usually in infancy for the first month of life because of recurrent severe infections. Beside a decrease of granulocytes, there is usually an increase in blood monocytes from two or four times over normal and increase in the blood eosinophil count is also common. The bone marrow analysis usually shows a maturation arrest of neutrophil precursors at the promyelocyte/myelocyte stage independent of the inheritance subtype. It was recently described that individuals treated with G-CSF had an increased incidence of MDS/AML over the years and, intriguingly, the risks of MDS-AML increase with the dose of G-CSF.

Further, it was demonstrated that myeloid cells from all patients with congenital neutropenia have reduced expression in LEF-1 transcription factor, suggesting that LEF-1 defect may be a common downstream defect. The myeloid cells from patients with SCN demonstrate an increased degree of apoptosis suggesting that defective or increased expression are mutation of one of the apoptosis-regulating genes could be the cause of neutropenia in some cases.

Granulopoiesis as part of the myelopoiesis, see FIG. 4e showing the various lineages and stages of myelopoiesis, is a life-long multistage process with continues generation of large number of mature neutrophils (>10⁶ cells/minute/kilogram bodyweight) from a small number of hematopoietic stem cells. To maintain continuous constitutive granulopoesis, all these events must be closely regulate by varieties of intrinsic transcription factors, such as RUNX, PU.1, C-EBPalpha and C-EBPbeta and distinct cytokines (e.g. G-CSF, GM-CSF, IL-3).

LEF/TCFs (T-cell factors) are a family of transcription factors regulated by the canonical Wnt signalling pathway and generally act in transcriptional complexes with β-catenin. LEF-1 is also known to act independently of β-catenin, for example, in the TGF-β and Notch pathways. LEF-1 belongs to the LEF-1/TCF family of high mobility group domain containing transcription factors. Although the gene structure of all these family members is remarkably similar, characterization of the full-length human LEF-1 gene locus and its complete set of mRNA products showed that LEF-1 exists as a unique set of alternatively spliced isoforms, and is functionally different from other TFCs. In addition, recent studies have described a dominant-negative LEF-1 isoform that lacks the β-catenin binding domain and functions as either a transcriptional repressor or transcriptional activator. Today, the analysis of the role of LEF-1 in hemapoiesis has been mostly restricted to the lymphoid compartment.

It was shown that LEF-1 is highly expressed in pro- and pre B-cells and thymocytes and is down-regulated in mature lymphocytes in a mouse model. LEF-1 has a context-dependent activation domain and in complex with its co-activator, ALY, contributes to maximum function of the T-cell receptor alpha enhancer in T-cell precursors independent of β-catenin. Binding of LEF-1 and activation of the RAG-2-promoter together with c-Myb and Pax-5 have been demonstrated. LEF-1 is expressed in premature B-cells and in premature and mature T-cells.

In the art, there is still an ongoing demand on providing suitable means for diagnosing and treating cytopenia, in particular, cytopenia of the lymphoid and myeloid lineage, in particular, for treatment of neutropenia, especially severe congenital neutropenia. Further, there is still a need for alternatives in the treatment of cancerous diseases or disorders, for example, of all types of leukemia.

Surprisingly, the present inventors found that LEF-1 is not only involved in the maturation of the lymphoid cell types but also of the cells of the myeloid lineage.

### SUMMARY OF THE INVENTION

In a first aspect, the present invention provides a method for diagnosing cytopenia in an individual, comprising the steps of a) determining the relative or absolute amount of LEF-1 or a functional fragment thereof; b) comparing the result of a) with the result determined using a control sample from a healthy individual or with an already known reference value allowing to determine the presence of absence of cytopenia in said individual.

Preferably, said cytopenia is a cytopenia of the myeloid lineage, in particular, neutropenia, especially severe congenital neutropenia.

In a further aspect, the present invention relates to the use of LEF-1 or a functional fragment or homolog thereof, or an enhancer or an inducer of LEF-1 expression, activity or LEF-1 mediated signalling for the preparation of a pharmaceutical for preventing or treating all types of cytopenia of the myeloid or lymphoid lineage. In particular, the present invention relates to the use of LEF-1, preferably, in its protein or nucleic acid form for preventing or treating neutropenia, in particular, severe congenital neutropenia.

In another embodiment, the present invention relates to a method of treating an individual suffering from cytopenia, in particular, of neutropenia, like severe congenital neutropenia, comprising the step of administering a therapeutically effective amount of LEF-1 or a functional fragment or homolog thereof, or an enhancer or an inducer of LEF-1 expression, activity or LEF-1 mediated signalling to an individual afflicted with cytopenia.

Another preferred embodiment of the present invention relates to the provision of a pharmaceutical composition comprising as active ingredients therapeutically effective amounts of LEF-1 or a functional fragment or homolog thereof, or an enhancer or an inducer of LEF-1 expression, activity or LEF-1 mediated signalling in combination with G-CSF, and, optionally, a pharmaceutical acceptable carrier.

In addition, the present invention provides a method for treating or preventing cancer, in particular, leukemia (myeloid and lymphoid, acute and chronic) using an inhibitor of LEF-1 expression or an antagonist of LEF-1 or a compound interacting with the LEF-1 signalling pathway in a cell.

In preferred embodiments, said inhibitor is a nucleic acid probe corresponding to the mRNA sequence encoding LEF-1, e.g. siRNA, shRNA, miRNA, antisense nucleic acid molecules or RNAi, as well as to small molecules or peptides acting as LEF-1-specific inhibitors.

Further, the present invention relates to methods determining the status of individuals undergoing clinical studies comprising the determination of the LEF-1 level to identify persons at risk of developing cytopenia, or leukemia.

Finally, the present invention relates to a method for mobilizing stem cells, in particular human stem cells and/or inducing differentiation or expansion (proliferation) of said stem cells or other pluripotent progenitor cells into the myeloid lineage comprising contacting said cells with LEF-1 or a functional fragment or homolog thereof, or an enhancer or an inducer of LEF-1 expression, activity or LEF-1 mediated signalling.

### BRIEF DESCRIPTION OF THE DRAWINGS

FIG. 1: FIG. 1 shows that the expression of LEF-1 and LEF-1 target genes is abrogated in CN myeloid precursors and is up-regulated in LEF-1 rescued CN CD34+ cells.

(a) LEF-1 mRNA expression in cells from different stages of myeloid differentiation. Individual cells were isolated (100 cells of each group) by laser-assisted single cell picking from bone marrow (BM) smears. Data represent means±s.d. of triplicates, studied values all P<0.05;
(b) Representative images of IF staining with LEF-1-specific antibody and DAPI (nuclei) of CD33+ BM myeloid progenitors of congenital neutropenia (CN) affected patients before and during G-CSF therapy (CN), one long-term G-CSF treated patient with cyclic neutropenia (CyN), and one healthy G-CSF-treated control (Control);
(c) mRNA expression of CD33+ cells of studied individuals, IN idiopathic neutropenia, Ctrl: control;
(d) mRNA expression of LEF-1 (lv)-, control (ctrl)-, or mock-transduced CN CD34+ cells (n=2) measured on day four of culture with or without G-CSF. Data represent means±s.d. and derived from three experiments each in triplicate (*, P<0.05; **, P<0.01), AU arbitrary units.

FIG. 2: FIG. 2 provides the restoration of defective LEF-1 expression promotes granulocytic differentiation of CN CD34+ progenitors. LEF-1 induces C/EBPalpha expression via direct binding to the C/EBPalpha promoter.

(a) Cell numbers, granulocytic (CD 15) surface marker expression after culture with G-CSF, and myeloid (CD 11b) surface marker expression after culture without G-CSF, of CD34+ SCN cells transduced the indicated constructs (n=2). Viable cell numbers were measured by trypan blue dye exclusion surface marker expression was measured by FACS;
(b) Wright-Giemsa staining of LEF-1 lv- or Crtl.-transduced CD34+ CN cells on day 12 of differentiation;
(c) C/EBPalpha mRNA expression of SCN CD34+ cells transduced with the indicated constructs (n=2) measured after 12 h of culture. AU: arbitrary units. Data represent means±s.d. and derived from three experiments each in triplicate; *, P<0.05; **, P<0.01;
(d) The LEF-1 binding site at the position from −559 bp to −538 bp of the 566 bp C/EBPalpha promoter using the NoShift competitor assay, which measured LEF-1 DNA-binding activity in nuclear extracts (NE) of CD34+ and CD33+ BM cells. Biotinylated oligonucleotides corresponding to the wild type sequence shown were used in the assay (WT), or used together with nonbiotinylated oligonucleotides with a mutant LEF-1 consensus-binding motif (WT/Mut); or nonbiotinylated LEF-1-specific competitor (WT/specific competitor); or nonbiotinylated oligonucleotides without LEF-1 consensus sequence as a nonspecific competitor (WT/nonspecific competitor) (*, P<0.05; n=3).
(e) ChIP assay of NE from CD34+ and CD33+ BM cells. PCR products were amplified using primer pairs flanking the LEF-1 binding site of the C/EBPalpha promoter. No Ab: no antibody, isotype, isotype antibody control.

FIG. 3: Anti-proliferative and pro-apoptotic effects of LEF-1 inhibition in CD34+ progenitors

CD34+ cells from healthy individuals (n=3) ansduced with LEF-1 shRNA (LEF-1 shRNA 975), control shRNA (ctrl shRNA gl4) and β-catenin shRNA (β-catenin shRNA 602) all containing an RFP reporter.
(a) mRNA expression of indicated genes in transduced cells measured on day four posttransduction; inset: western blot analysis of LEF-1 protein expression in total lysates of CD34+ cells, transduced with control and LEF-1 shRNA;
(b) β-catenin mRNA and protein expression (inset) in CD34+ cells transduced with control or β-catenin shRNA;
(c) proliferation of sorted RFP+ cells was determined by counting RFP+BrdU+ double-positive cells by FACS on day four post-transduction.
(d) Viable cells were counted using trypan blue dye exclusion;
(e) percentage of apoptotic cells (Annexin V FITC+ RFP+) on day four posttransduction;
(f) LEF-1 shRNA transduced sorted CD34+ cells (10E3 cells/well) were treated with 10, 100, 1000 ng/ml of GM-CSF or G-CSF and counted monocytes/macrophages on Wright-Giemsa-stained cytospin slides on day seven of culture; (a-d, f) data represent means±s.d. and derived from two experiments each in duplicate; *, P<0.05; **, P<0.01.

FIG. 4: LEF-1 overexpression in CD34+ cells promoted up-regulation of LEF-1 target genes and increased proliferation. CD34+ cells from healthy individuals (n=3) were transduced with LEF-1 lv, and ctrl lv, positively sorted and cultured.

(a, b) mRNA expression levels of indicated genes on day four posttransduction; inset in (a) represents Western blot analysis of total lysates of CD34+ cells transduced with ctrl lv and LEF-1 lv; AU arbitrary units;
(c) proliferation of sorted GFP+ cells by counting GFP+BrdU+ double-positive cells by FACS on day four post-transduction;
(d) Viable cells were counted using trypan blue dye exclusion; data represent means±s.d. and derived from three experiments each in triplicate (*, P<0.05; **, P<0.01);
(e) schematic model of the stage-specific and lineage-specific functions of LEF-1 in hematopoiesis. LEF-1 induces granulocytic differentiation of promyelocytes (box), similar to its effects in lymphocyte differentiation at the pre-mature stage (arrows); (f) effects of LEF-1 on the initiation of the myelopoietic maturation program are mediated via the regulation of distinct target genes.

FIG. 5: FIG. 5 provides the mRNA expression of secretory granule proteins; LEF-1 mRNA/protein expression as well as TCF-3,-4 mRNA expression

(a) mRNA expression of secretory granule proteins (MPO and MMP9) in BM cells of different stages of myeloid differentiation. Individual cells (100 cells/group) were isolated by laser-assisted single cell picking from BM smears. Data represent means±s.d. of duplicates;
(b) LEF-1 protein expression in CD34+ cells. Western blot analysis of total lysates of CD34+ and Jurkat cells (positive control) using LEF-1-specific rabbit polyclonal (LEF-1 pAb) or mouse monoclonal (LEF-1 mAb REMB1) antibody. LEF-1 mRNA (c) and protein (d) expression in peripheral blood CD14+ monocytes and CD3+ lymphocytes of CN patients (n=14) and healthy controls (n=5).
(e) TCF-3, TCF-4 mRNA expression in BM CD33+ cells of studied individuals. Data represent means±s.d. *, P<0.05; **, P<0.01.

FIG. 6: FIG. 6 shows the mRNA expression of indicated genes in sorted GFP-CD34+ CN cells from the LEF-1 lv and dnLEF-1 lv experiments mRNA expression levels of sorted GFP-cells and mock samples were compared using qRT-PCR. Data represent means±s.d. and derived from three experiments each in duplicate.

FIG. 7: FIG. 7 demonstrates the mRNA expression of indicated genes in sorted RFP-cells from the shRNA transduction experiments. Morphology of LEF-1 and β-catenin shRNA transduced cells (a) mRNA expression levels of sorted RFP-cells and mock samples were compared using qRT-PCR. Data represent means±s.d. and derived from two experiments each in duplicate;

(b) morphological evidence of apoptotic cells (black arrows) on cytospin preparates of LEF-1 shRNA transduced cells.

FIG. 8: FIG. 8 shows the effects of LEF-1 inhibition in two myeloid cell lines HL-60 and K562

LEF-1 expression either in HL-60 or K562 cells was inhibited by transduction of LEF-1 shRNA (LEF-1 shRNA 975) and control shRNA (ctrl shRNA gl4) with a RFP reporter. Additionally, K562 cells were transduced with β-catenin shRNA (β-catenin 602). RFP+ cells were sorted and measured
(a) mRNA expression of indicated genes by qRT-PCR on day four post-transduction; insets: Western blot analysis of LEF-1 protein in K562 and HL-60 cells transduced with LEF-1 shRNA and control construct.
(b) Western blot analysis of β-catenin protein expression in K562 myeloid cell line transduced with β-catenin shRNA; control sample is presented with three increased concentrations of protein.
(c,d) proliferation of sorted RFP+ cells was determined by counting of viable cells using trypan blue dye exclusion and by (d) BrdU incorporation and counting of RFP+BrdU+cells by FACS on day four post-transduction. Data represent means±s.d. and derived from three experiments each in duplicate. *, P<0.05; **, P<0.01.
Apoptosis in RFP+ sorted cells was analysed using (e) Annexin V-FITC staining and (f) apparent morphology (black arrows) on day four post-transduction.

FIG. 9: FIG. 9 provides the mRNA expression of indicated genes in sorted RFP-cells from the shRNA transduction experiments in K562 and HL-60 cell lines

(a) mRNA expression levels of sorted RFP-cells and mock samples of K562 and HL-60 cells were compared using qRT-PCR. Data represent means±s.d. and derived from two experiments each in duplicate.

FIG. 10: FIG. 10 demonstrates the mRNA expression of indicated genes in sorted GFP-cells from the experiments in LEF-1 lv transduced CD34+ cells of healthy controls (a) mRNA expression levels of sorted GFP-cells and mock samples were compared using qRT-PCR. Data represent means±s.d. and derived from three experiments each in duplicate.

FIG. 11: FIG. 11 shows the data for the analysis of proliferation arrest and apoptosis of AML cells after transduction with shRNA specific for LEF-1.

### DETAILED DESCRIPTION OF THE PRESENT INVENTION

The present inventors recognized that LEF-1 is crucial inter alia for neutrophil granulocytopoesis and, e.g., its expression is severely reduced in cytopenia, in particular, cytopenia of the myeloid and lymphoid lineage, like in neutropenia, in particular in severe congenital neutropenia.

Thus, in a first aspect, the present invention relates to a method for diagnosing cytopenia involving the step of a) determining the relative or absolute amount of LEF-1 or a functional fragment thereof expression in sample of an individual and b) comparing said relative or absolute amount with a reference example from a healthy individual or a reference value determined before allowing to identify persons suffering from cytopenia.

In a further embodiment, the present invention relates to the use of LEF-1, a functional fragment or homolog thereof, or an enhancer or an inducer of LEF-1 expression, activity or LEF-1 mediated signalling for the preparation of a pharmaceutical for preventing or treating cytopenia in an individual suffering therefrom.

In a preferred embodiment said cytopenia is a cytopenia of the lymphoid or myeloid lineage. In particular, said cytopenia is a cytopenia of the myeloid lineage, in particular, neutropenia. Especially preferred, said cytopenia is severe congenital neutropenia.

In addition, the present invention relates to a method for preventing or treating cytopenia of the myeloid or lymphoid lineage in an individual comprising the step of administering LEF-1 or a functional fragment or homolog thereof, or an enhancer or an inducer of LEF-1 expression, activity or LEF-1 mediated signalling in a therapeutically effective amount to an individual in need thereof. In particular, said method relates to preventing or treating cytopenia of the myeloid lineage, like neutropenia, in particular, severe congenital neutropenia.

The LEF-1 molecule may be in a form of a protein, e.g. according to SEq.-ID No. 2 or peptide or in form of a nucleic acid molecule, e.g. Seq.-ID No. 1. In a preferred embodiment the inducer or enhancer of LEF-1 expression, activity or LEF-1 mediated signalling is a molecule enhancing transcription of nucleic acid molecules encoding LEF-1 or a molecule stabilizing LEF-1 and extending the half-life of the protein or stabilizing mRNA in the cell.

In the method for preventing or treating cytopenia as described above, optionally, a therapeutically effective amount of G-CSF is administered to said individual. The combination of LEF-1 with G-CSF allows to increase the numbers of granulocytes and/or to decrease required therapeutic doses of G-CSF in the individual suffering from cytopenia, in particular, neutropenia. Particularly, this is of economic and therapeutic interests since treatment with G-CSF requires daily injection of G-CSF and the medical costs for G-CSF treatment are very high.

In another embodiment, the present invention relates to the use of an inhibitor of LEF-1 expression and/or LEF-1 mediated signalling or an antagonist of LEF-1 alleviating or interrupting the down-stream signalling of LEF-1 in a cell.

In particular, the inhibitor or antagonist allows treating or preventing ALL, AML or CML. In case of myeloid leukemias, the inhibitor of LEF-1 expression or LEF-signalling enables treating cancer of the myeloid lineage with transformed myeloid precursors, in particular, acute myeloblastic leukaemia, acute promyelocytic leukaemia, acute myelomonocytic leukaemia and acute monoblastic leukaemia as well as chronic myeloblastic leukaemia.

That is, on the one hand, the active ingredient of the pharmaceutical preparation according to the present invention for preventing or treating cancer of the myeloid or lymphoid lineage is a molecule interacting with the nucleic acid molecules encoding LEF-1 protein, e.g. Seq.-ID No. 2 for human LEF-1, on DNA or mRNA level or, on the other hand, is a molecule which decreases or interrupt transcription of the nucleic acid molecule encoding LEF-1 or alleviating or interrupting down-stream signalling of LEF-1, i.e. an antagonist of LEF-1 or a molecule intercepting with the LEF-1 protein, thus, inhibiting the formation of activation complexes formed by LEF-1 with their respective binding partners to activate e.g. c-myc, survivin or cyclin-D1.

It has been recognized by the present inventors that LEF-1 plays a crucial role in inter alia granulocytopoiesis, in particular of neutrophil granulocytopoiesis. Thus, on the one hand, interrupting the LEF-1 signalling pathways allows reducing or inhibiting excessive proliferation, survival and differentiation of granulocyte progenitor cells. On the other hand, fostering and elevating the LEF-1 level in combination with corresponding growth factors (e.g. G-CSF to induce granulopoiesis) enables overcoming cytopenia of the myeloid and lymphoid lineage, in particular, overcoming a maturation arrest in the differentiation of progenitor cells of granulocytes.

It has been shown that overexpression of LEF-1 in CD34+ progenitor cells without addition of any differentiation-inducing cytokine increased proliferation of these cells without differentiation. This data suggest that very high LEF-1 expression levels may lead to enforced proliferation of hematopoietic progenitors, as in case of leukaemia.

In another preferred embodiment, the present invention relates to a method for the differentiation of different types of cytopenia comprising the step of determining the expression of LEF-1 in a probe of an individual suspected to suffer from a cytopenia. Preferably, said method relates to the diagnosis of severe congenital neutropenia.

The present invention is also useful for the stratification of the treatment of cytopenia and cancer, like leukemia. That is, the present invention allows monitoring the therapy of any type of cytopenia as described herein or of monitoring the therapy of a cancer of the myeloid or lymphoid lineage characterized in having increased LEF-1 expression.

That is, by determining the expression of the LEF-1 protein during therapy it is possible to determine the absolute neutrophil count, thus, identifying the therapeutical success of the regimen.

The term “control sample” refers to a sample of an individual of the same species which individual is not suffering from the specified disease or disorder. Preferably, the control sample is of the same type, as the sample, for example, the sample and the control sample are both plasma samples or are both tissue samples etc.

With the term “reference value” as used throughout the specification, a reference value is meant which is a known value of the LEF-1 molecule to be determined in the diagnostic or stratification method. The reference value can be determined prior, simultaneous with or after the value of the sample has been determined.

### “Sample/Probe”

A “sample/probe” according to the invention is biological material which has been obtained from an individual, such as whole blood, plasma, serum, hemofiltrate, urine, bone marrow, bone marrow plasma or serum, bone marrow biopsy, or tissue. A sample or probe can also be a material indirectly obtained from an individual, such as cells obtained from the individual which may have been cultured in vitro prior to obtain the sample from these in vitro culture cells which can be the cells itself or the cell culture supernatant obtained from these cells. A sample or probe can also be pretreated prior to analysis with the methods of the invention. Cited pretreatments for example can be storage of the sample at various temperatures, such as room temperature, 4° C., 0° C., −20° C., −70° C., −80° C., or at other temperatures, or storage on water ice or dry ice or storage in liquid nitrogen or storage in other solid, liquid or gas media. Further pretreatments among others are filtration of the sample, precipitation of the sample in using salts, organic solvents, such as ethanol or other alcohols, acetone etc., separation of the sample into subfraction using methods such a chromatography, liquid phase extraction, solid phase extraction, immune precipitation using antibodies, antibody fragments or other substances binding to constituents of the sample. Chromatography methods among others are size exclusion chromatography, anion or cation chromatography, affinity chromatography, capillary chromatography, etc., for example reverse phase chromatography.

The term “amount” is meant to describe the absolute amount of a protein, peptide or nucleic acid molecule or the relative amount of a peptide, protein or nucleic acid molecule relative to for example the same peptide, protein or nucleic acid molecule in a control sample or relative to the reference value of the same peptide, protein or nucleic acid molecule. “Relative” means, that not distinct amounts such as mole or milligram per litre etc. are stated, but that for example is stated that the sample contains more, less or the same amount of a certain peptide, protein or nucleic acid as compared to a control sample or reference value. The term “more, less or the same amount” in this situation includes also, if only measurement units are stated, such as absorption value, extinctions, coefficients, mass spectrometric signal intensities, densitometric measurements or Western Blot, or other types of measurement values, which do not translate into absolute amounts of a peptide, protein or nucleic acid molecule.

As used herein, the term “individual” or “subject” is used herein interchangeably and refers to an individual or a subject in need of a therapy or prophylaxis or susceptive to be afflicted with a condition or disease mentioned herein. Preferably, the subject or individual is a vertebrate, even more preferred a mammal, particular preferred a human.

The term “a functional fragment thereof” as used herein refers to a fragment of the LEF-1 protein, i.e. a peptide, or the nucleic acid encoding LEF-1, e.g. the nucleic acid according to Seq.-ID-No.1 which display the same activity. In the present document, the term LEF-1 includes LEF-1 fragments unless otherwise indicated. Further, unless explicitly noted, the term LEF-1 includes salts or solvates of the compounds. E.g. the LEF-1 protein is the protein according to Seq.-ID-No. 2.

The term “homolog” as used herein refers to molecule having LEF-1 activity. In particular, a homolog has the same activity as LEF-1 in the LEF-1 mediated signalling pathway. In the present document, the term LEF-1 includes LEF-1 homologs which may be present in a salt or solvate form unless otherwise indicated.

Activity of LEF-1 may be determined by methods known in the art, e.g. by PCR, western blot, northern blot, ELISA, reporter gene assay of LEF-1 target genes.

The pharmaceutical composition according to the present invention comprises the LEF-1 molecule or an inducer or enhancer of the LEF-1 molecule as described herein or in case of a pharmaceutical composition for treating cancer, the inhibitor or antagonist of LEF-1 as described herein and, optionally, a pharmaceutical acceptable carrier. Such pharmaceutical compositions comprise a therapeutically effective amount of the conjugates and, optionally, a pharmaceutically acceptable carrier. The pharmaceutical composition may be administered with a physiologically acceptable carrier to a patient, as described herein. Acceptable means that the carrier be acceptable in the sense of being compatible with the other ingredients of the composition and not be deleterious to the recipient thereof. In a specific embodiment, the term “pharmaceutically acceptable” means approved by a regulatory agency or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium, carbonate, etc. Examples of suitable pharmaceutical carriers are described in “Remington's Pharmaceutical Sciences” by E. W. Martin (18^{th} ed., Mack Publishing Co., Easton, Pa. (1990)). Such compositions will contain a therapeutically effective amount of the aforementioned compounds, salts or solvates thereof, preferably in purified form, together with a suitable amount of carrier so as to provide the form for proper administration to the patient. The formulation should suit the mode of administration.

Typically, pharmaceutically or therapeutically acceptable carrier is a carrier medium which does not interfere with the effectiveness of the biological activity of the active ingredients and which is not toxic to the host or patient.

In another preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in a unit dosage form, for example, as a dry lyophilised powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical composition for use in connection with the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The term “therapeutically or pharmaceutically effective amount” as applied to the compositions of the present invention refers to the amount of compositions sufficient to induce the desired biological result. That result can be alleviation of the signs, symptoms or causes of a disease or any other desired alteration of a biological system. In the present invention, the result will typically involve e.g. decrease or increase of LEF-1 expression and, thus, altering the absolute neutrophil count in said individual.

In vitro assays may optionally be employed to help identifying optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. Preferably, the pharmaceutical composition is administered directly or in combination with an adjuvant

The term “administered” as used herein means administration of a therapeutically effective dosage of the aforementioned pharmaceutical composition to an individual. By “therapeutically effective amount” is meant a dose that produces the effects for which it is administered. The exact dose will dependent on the purpose of the treatment and will be ascertainable by once skilled in the art using known techniques. It is known in the art adjustments for systemic vs. localized delivery, age, body weight, general health, sex, diet, time of administration, drug interaction and severity of the condition may be necessary, and will be ascertainable with routine experimentation by those skilled in the art.

The administration of the pharmaceutical composition can be done in a variety ways including, but not limited to orally, subcutaneously, intravenously, intraarterial, intranodal, intramedulary, intradecal, intraventricular, intranasaly, intrabronchial, transdermally, intrarectally, intraperitonally, intramuscularly, intrapulmonary, vaginaly or intraoculary.

The attending physician and clinician will determine the dosage regimen. A typical dose can be, for example, in the range of 0.001 to 1000 μg; however, doses below or above this exemplary range are envisioned, especially considering the aforementioned factors.

As indicated before, in one embodiment, the present invention relates to a method for the pharmacogenetic analysis of persons undergoing treatment with a (tumor) therapeutic drug or other drugs to be tested in pharmacogenetics studies in clinical trials comprising the step of determining the level of LEF-1 expression in said individual. The determination of the LEF-1 expression level allows identifying individuals being at risk of developing a cytopenia, in particular, neutropenia as well as allowing detecting the presence of pre-leukemic syndromes in the individual participating clinical trials. In addition, the determination of the LEF-1 status of an individual undergoing clinical trials would reveal individuals having or developing elevated LEF-1 levels, thus, being at risk of developing leukaemia.

Further, it is possible to determine a myelodysplastic syndrome/pre-leukemic syndrome in an individual comprising the step of determining the expression of LEF-1 in said individual.

Thus, the present invention allows to treat leukaemia and other malignancies associated with elevated LEF-1 levels by inhibiting LEF-1 mRNA/protein synthesis, or blocking of LEF-1 protein using e.g. LEF-1-specific small inhibitory molecules, inhibitory synthetic peptides or inhibitory RNAs or DNAs or similar nucleic acid like molecules like PNA etc.

In addition, according to the present invention, methods are provided for the treatment of congenital non-malignant disorders of hematopoiesis, e.g. congenital neutropenia, immunodeficiencies due to altered expression and activity of LEF-1. Said methods involve the use of LEF-1-specific stimulatory small molecules of stimulatory synthetic peptides.

Another aspect of the present invention relates to the use of LEF-1, or a fragment or homolog thereof, or an inducer or enhancer of LEF-1 expression, activity or LEF-1 mediated signalling in the mobilisation and differentiation of stem cells in particular of human stem cells in a fashion similar to the way it is described and well-known in the literature for G-CSF.

These and other embodiments are disclosed and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.tigr.org/, are known to the person skilled in the art and can also be obtained using, e.g., http://www.google.de. An overview of patent information in biotechnology and a survey of relevant sources of patent information useful for retrospective searching and for current awareness is given in Berks, TIBTECH 12 (1994), 352-364.

Not to be bound by theory, the present invention will be described further by the way of examples. It is clear that said examples are intended to illustrate the present invention further without limiting the invention thereto.

### EXAMPLES

The following methods have been applied in the present invention:

To study the regulation of myelopoiesis, initially mRNA expression patterns of different transcription factors in CD33⁺ myeloid progenitors (which consists predominantly of promyelocytes) from individuals with CN in comparison to healthy controls are characterized.

Participants in this study included: thirteen CN patients; four CyN patients (CyN); four patients with neutropenias associated with congenital disorders of metabolism (MN)-one patient suffered from a glycogen storage disease type Ib, three patients from Shwachman-Diamond Syndrome; two patients with idiopathic neutropenia (IN) all long-term G-CSF-treated (>one year); as well as two CN patients prior to G-CSF therapy, and three healthy volunteers (ctrl) received G-CSF in a dose of 5 μg/kg/day for two days. We collected BM samples in association with the annual follow-up recommended by the Severe Chronic Neutropenia International Registry (SCNIR) and informed consent was obtained from all subjects.

### Cell Purification and Separation.

BM and blood mononuclear cells were isolated by Ficoll-Hypaque gradient centrifugation (Amersham Biosciences) and positively selected BM CD34+, CD33+, and blood CD14+, CD3+ cells using sequential immunomagnetic labeling with corresponding MACS beads (Miltenyi Biotech). For shRNA experiments, G-CSF primed peripheral blood CD34+ cells were used.

Quantitative Real-Time RT-PCR (qRT-PCR).

For qRT-PCR, we isolated RNA using QIAGEN RNeasy Mini Kit (Qiagen) or TRIZOL reagent (Invitrogen) using manufacturer's protocol with slight modifications (see below), amplified cDNA using random hexamer primer (Fermentas) and measured mRNA expression using SYBR green qPCR kit (Qiagen).

Target gene mRNA expression was normalized to β-actin and was represented as arbitrary units (AU). As primers, primers according to Seq.-ID-Nos. 3 to 6 may be used. LEF-1 mRNA primers (Seq.-ID-No. 3 and 4 or 5 and 6, respectively) detected both full length and dnLEF-1.

### Laser-Assisted Cell Picking.

Cells of BM slides were isolated using the PALM Laser-MicroBeam System (P.A.L.M.) and controlled the purity of individual populations (100 cells/sample) by qRT-PCR of myeloid-specific primary (myeloperoxidase; MPO) and secondary (matrix metalloproteinase 9; MMP9) granule proteins (FIG. 5a).

### Confocal Fluorescence Microscopy.

1×10E5 CD33+ cells were fixed on slides in 4% paraformaldehyde for 10 min, permeabilized with 0.5% Triton X-100 for 10 min, incubated with LEF-1-specific rabbit polyclonal antibody (1:2,000 dilution) for one h at 37° C., with secondary FITC-conjugated antibodies for 30 min. Nuclei were counterstained with DAPI.

### Analysis of LEF-1 Binding to the C/EBPa Promoter in Nuclear Extracts of CD34+ and CD33+ BM Cells.

Competitive biotinylated transcription factor oligonucleotide binding NoShift assay (Novagen) was used, which is a colorimetric assay similar to an electrophoretic mobility shift assay (EMSA), and chromatin immunoprecipitation (ChIP) assay. Details are given below, see also table 1.

### Lentiviral Transduction of CD34+, K562 and HL-60 Cells.

Construction of LEF-1 cDNA, dnLEF-1 cDNA as well as shRNA-containing lentiviral vectors as described below. Recombinant lentiviral supernatants were prepared, as described previously (Scherr et al., Blood. 2006; 107(8):3279-87). The virus titers averaged and typically ranged between 1-5×10E8 IU/ml. CD34cells from three healthy donors, HL-60 and K562 cells (1×10/well) were transduced with lentiviral supernatants with a MOI (multiplicity of infection) of 1-2, as described previously, re-transduced after 12-24 h and assessed transduction efficiency after 72 h as the percentage of GFP+, or RFP+ cells analyzed by FACS. Mock-transduced control virus-free conditioned medium from nontransfected cells was used as a control.

### In Vitro Proliferation and Granulocytic Differentiation Experiments.

1×10E5 of transduced and sorted RFP+ or GFP+ CD34+ cells were cultured in X-VIVO 10 medium (Cambrex) supplemented with 20 ng/ml of interleukin-3 (IL-3), 20 ng/ml of interleukin-6 (IL-6), 20 ng/ml of thrombopoietin (TPO), 50 ng/ml of stem cell factor (SCF), and 50 ng/ml of flt3-ligand (FLT-3l), all purchased from R&D Systems. We cultured HL-60 and K562 cells (1×10E5/well) in RPMI 1640 10% FCS supplemented medium. For assessment of proliferation viable cells were counted using trypan blue dye exclusion in haemocytometer and measured BrdU uptake using BrdU Flow Kit (Pharmingen). The percentage of apoptotic cells was determined using annexin V-FITC conjugate (Pharmingen) and by counting the apoptotic cells with morphological evidences of apoptosis (chromatin condensation and fragmented nuclei) on cytospin preparates. For granulocytic differentiation, 1×10E5 of GFP transduced CD34cells from two CN patients were cultured in supplemented RPMI 1640 1% FCS medium in the presence of G-CSF (10 ng/ml). Granulocytic differentiation was characterized by FACS analysis of cells stained with PE-conjugated CD15-specific (Caltag) and PE-conjugated CD11b-specific (Pharmingen) antibody and by morphological assessment of Wright-Giemsa-stained cytospin slides.

### Western Blot Analysis.

The following antibodies were used: mouse monoclonal LEF-1 (REMB1, Calbiochem), rabbit polyclonal LEF-1 antiserum, mouse monoclonal β-catenin (BD Transduction Laboratories), rabbit monoclonal β-actin and secondary anti-mouse or anti-rabbit HRP conjugated antibody all from Santa Cruz. Whole cell lysates was obtained either through lysis of a defined number of cells in lysis buffer or through direct disruption in Laemmli's loading buffer followed by brief sonication. Proteins were separated by 10% SDS-PAGE and the blots were probed either 1 h at 24° C. or overnight at +4° C.

### Statistical Analysis.

Statistical analysis was performed using the SPSS V. 9.0 statistical package (SPSS) and a two-sided unpaired Student's t test for the analysis of differences in mean values between groups.

### Gene Bank Accession Numbers.

Human LEF-1 NM_{—}016269; human β-catenin NM_{—}007614; C/EBPa promoter S75265.

mRNA Isolation from Cells Obtained by Laser-Assisted Single Cell Picking

mRNA was isolated using TRIZOL reagent (Invitrogen) according to the manufacturer's protocol with slight modifications: 10 ng/ml of tRNA and 50 ng/ml of linear polyacrylamide (LPA) (both Sigma-Aldrich) were added to the TRIZOL.

### Colorimetric Transcription Factor Oligonucleotide Binding Assay (NoShift Assay)

The competitive biotinylated oligonucleotide binding assay (Novagen) is a colorimetric assay similar to an electrophoretic mobility shift assay (EMSA). Double-stranded probes consisting or not of 3′-biotinylated oligonucleotides corresponding to the human LEF-1 binding site at the position from −559 bp to −538 bp of the known 566 bp C/EBPalpha upstream promoter (see Table 1) were annealed by heating to 100° C. for 10 min and cooling to room temperature. Annealed products were confirmed by agarose gel electrophoresis, incubated probes (10 pmol) with 20 μg of nuclear protein in a 20 μl reaction containing NoShift binding buffer, Poly(dI-dC)•Poly(dI-dC), and salmon sperm DNA for 30 min at 4° C. per the manufacturer's instructions. Reaction mixtures for 1 h at 37° C. were incubated in prewashed 96-well streptavidin-coated plates, washed wells three times for 5 min with wash buffer, incubated with primary LEF-1-specific antibody (1:1000 dilution) for 60 min at 37° C., washed again, and incubated with horseradish peroxidase (HRP)-conjugated secondary antibody to rabbit (30 min at 37° C., 1:1,000). HRP substrate (tetramethyl benzidine, 100 μl) was added after washing wells and absorbance (450λ) was determined. Similar to an EMSA assay, appropriate controls include competition studies with a 5-fold molar excess of non-biotinylated LEF-1-specific oligonucleotide, non-biotinylated oligonucleotide containing point mutations within the LEF-1 binding sequence or non-biotinylated oligonucleotide having no LEF-1 consensus sequence as a non-specific competitor.

### Chromatin Immunoprecipitation (ChIP) Assay

CD34+ and CD33+ cells (5×10E6) were cross-linked in 1% formaldehyde for 10 min at room temperature, stopped the cross-linking reaction by adding 0.125 M glycine, rinsed twice in ice cold PBS with protease inhibitors, re-suspended in 1 ml of SDS lysis buffer containing protease inhibitors, and incubated 10 min on ice. DNA-protein complexes were sonicated with three 15 s pulses at 50% of the maximum output. One-tenth of the sample was set aside for input control, the remaining sample was precleaned with blocked Staph A cells, immunoprecipitated precleaned chromatin using the LEF-1-specific polyclonal antibody and eluted immunoprecipitated protein-DNA complexes. The cross-links were reversed with 0.3 M NaCl at 67° C. for four h and deproteinated with 20 μg/ml of proteinase K (Invitrogen) in the presence of 0.5% SDS. LEF-1-associated DNA were detected by PCR amplification using 200 ng of immuno-precipitated DNA as well as 200 ng of 1/100 of total input and subsequent DNA sequencing using dye terminator method (ABI).

LEF-1 cDNA Synthesis and Construction of LEF-1 cDNA and dnLEF-1 cDNA Containing Lentiviral Vectors

1,220 bp LEF-1 cDNA were amplified and cloned into pRRL.PPT.SF.i2GFPpre vector. This vector is a derivative of the standard lentiviral vector pRRL.PPT.PGK.GFPpre (kindly provided by Luigi Naldini, Milano, Italy). To construct the dominant negative LEF-1 (dnLEF-1), pRRL.PPT.SF.LEF-1.i2GFPpre were cut with BamHl, treated with Klenow polymerase, redigested with BsrGI and ligated with a StuI/BsrGI fragment (containing the dnLEF-1 IRES GFP cassette) of the same vector.

shRNA Synthesis, Construction of LEF-1 shRNA and β-Catenin shRNA Expression Cassettes and shRNA Containing Lentiviral Vectors

DNA oligonucleotides were chemically synthesized corresponding to position 975 bp to 994 bp of the human LEF-1 gene sequence (Seq.ID-Nos. 7 and 8, respectively), and to position 602 bp to 620 bp of the human β-catenin gene sequence. These nucleotides also contained overhang sequences from a 5′ BglII- and a 3′ SalI-restriction sites (BioSpring). The numbering of the first nucleotide of the shRNAs refers to the ATG start codon. The oligonucleotides were inserted into the BglII/SalI-digested pBlueScript-derived pH1-plasmid to generate pH1-LEF-1 975 and the isolated clone by DNA was verified sequencing. The plasmid pH1-gl4 (control) is known in the art. To generate lentiviral transgenic plasmids containing H1-shRNA expression cassettes located in the U3 region of the Δ3′-LTR pdc-SR36 was used. To generate the lentiviral pdcH1-LEF-1-975-SR and pdcH1-β-catenin-602-SR plasmids, the pH1-LEF-1-975 as well as pH1-β-catenin-602 were digested with SmaI and HincII and ligated the resulting DNA fragments (360 bp) into the SnaBI site of the pdc-SR. The lentiviral plasmid encodes RFP_{EXPRESS} as reporter gene.

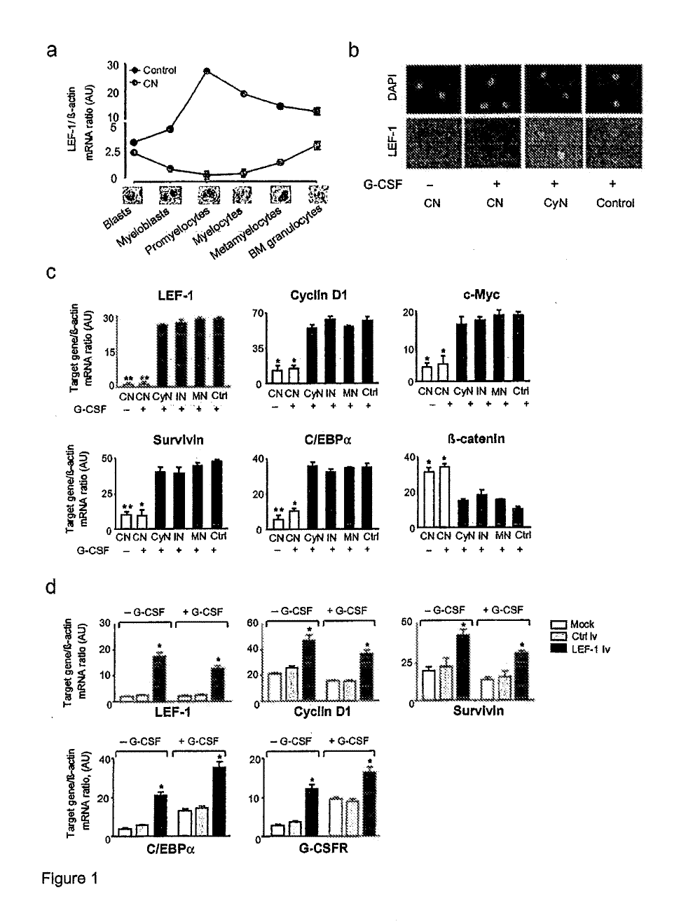

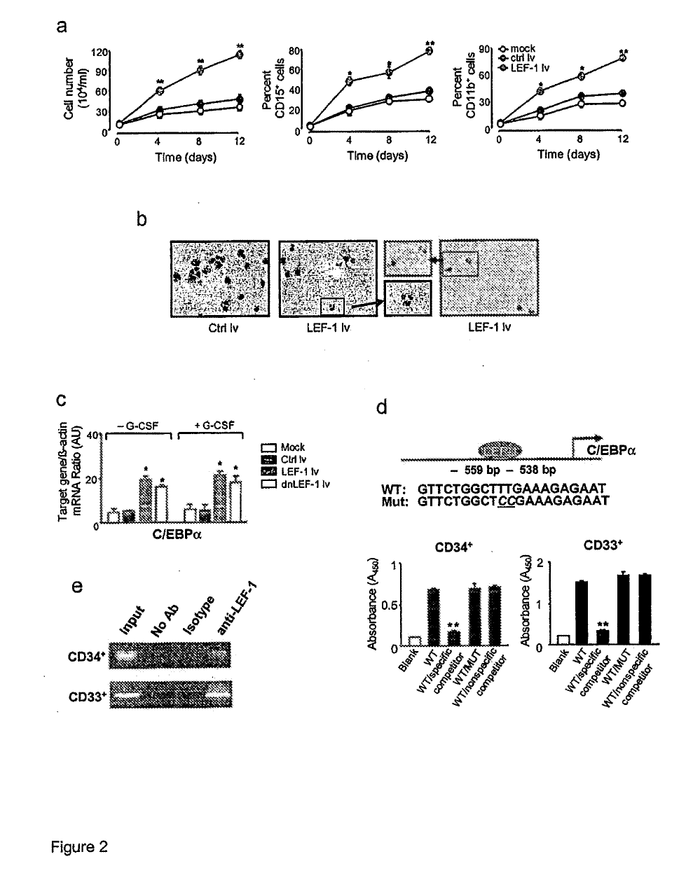

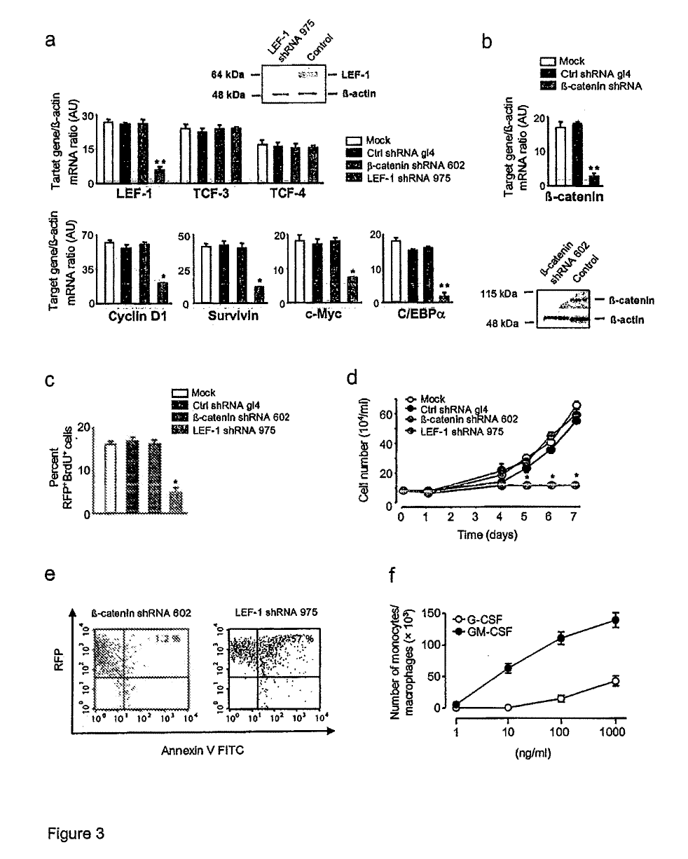

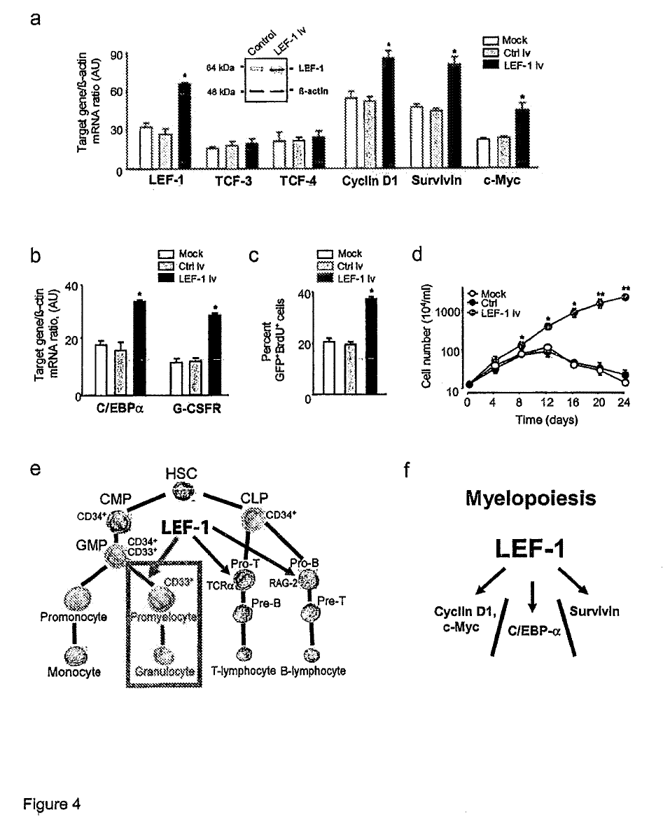

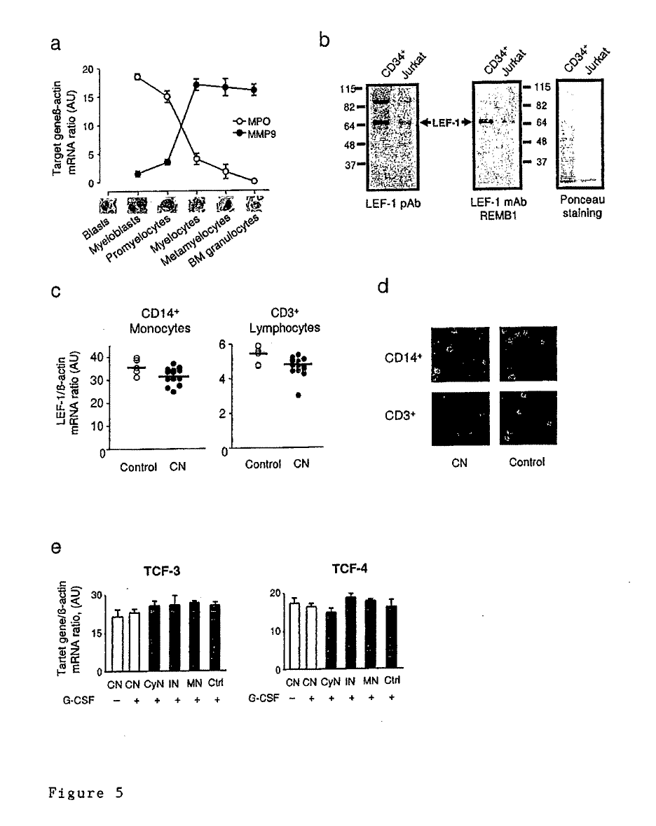

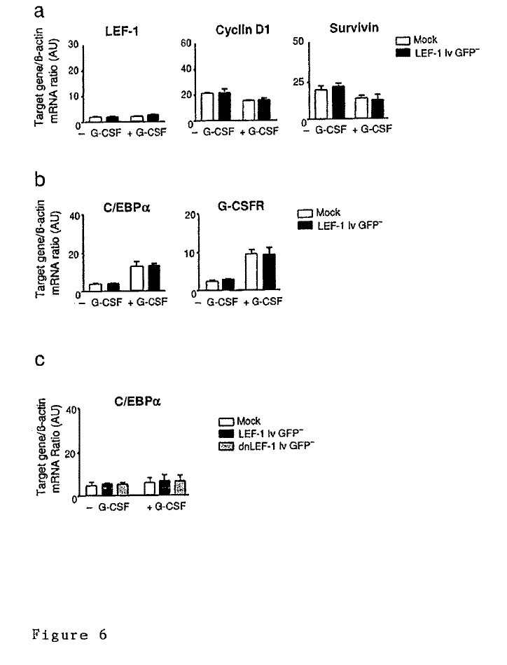

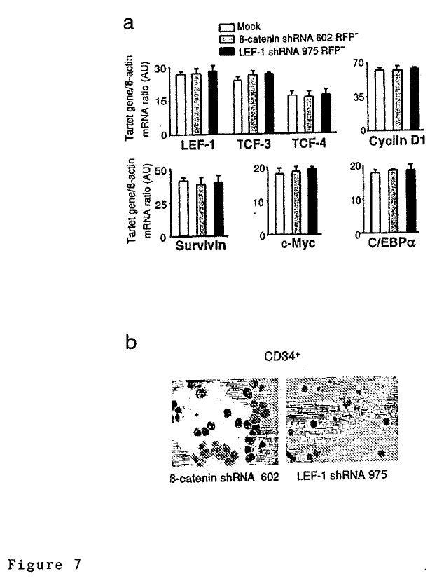

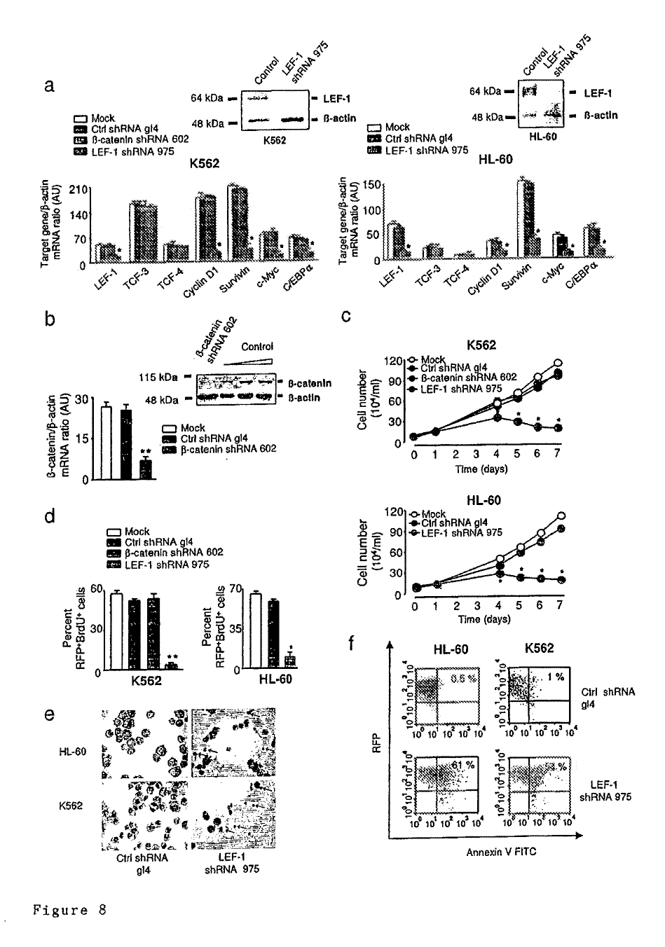

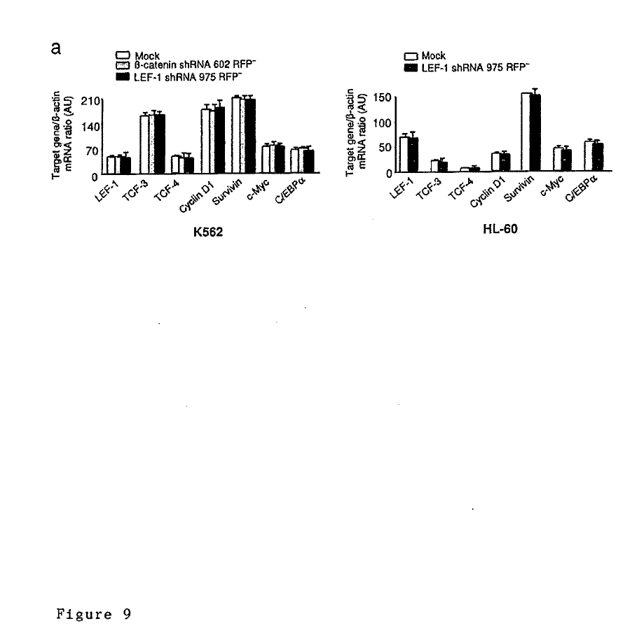

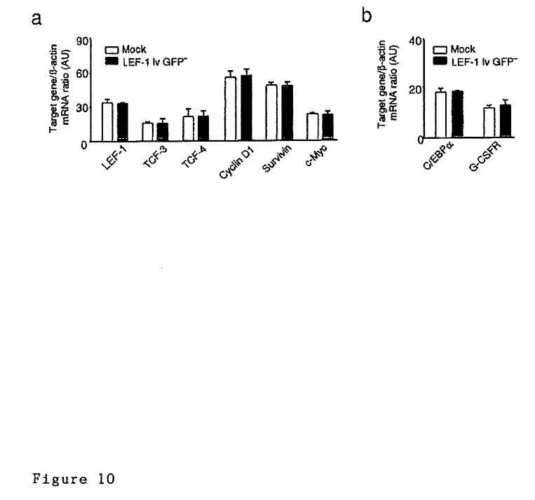

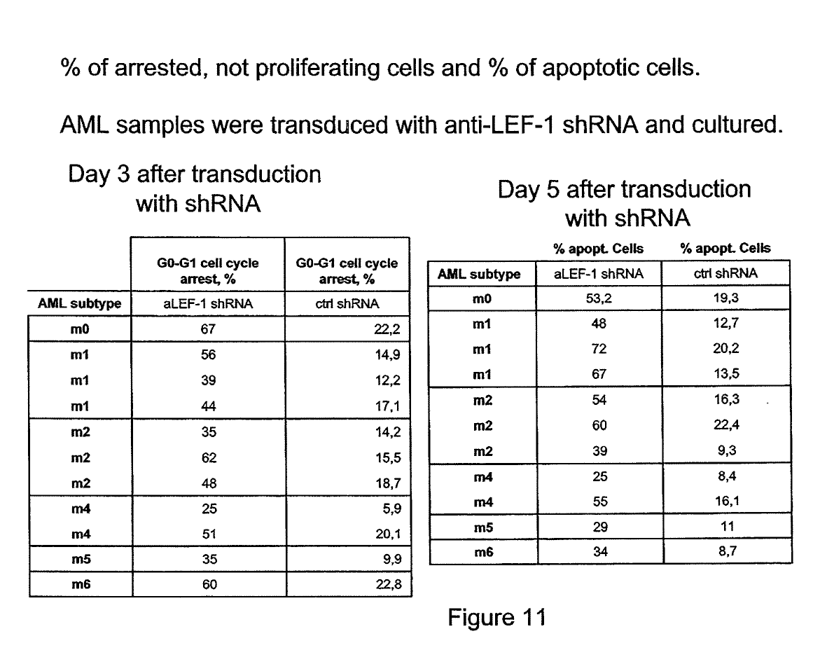

To understand the regulation of myelopoiesis, mRNA expression patterns of different transcription factors in CD33+ myeloid progenitors (which consist predominantly of promyelocytes) from SCN patients in comparison to healthy controls have been characterized. It was found that LEF-1 mRNA expression was 20-fold down-regulated or even completely absent only in SCN samples. Down-regulation of LEF-1 mRNA in SCN BM cells at different stages of myeloid differentiation isolated by laser-assisted single cell picking was demonstrated. In healthy controls, varying LEF-1 expression levels at all stages of myelopoiesis were found with peak expression in promyelocytes. LEF-1 mRNA expression in promyelocytes of SCN patients was significantly decreased (P<0.05) and in some cases completely absent (FIG. 1a). this was confirmed on the protein level by lack of fluorescent signal only in SCN CD33+ myeloid progenitors stained with a LEF-1-specific antibody (FIG. 1b, FIG. 5b). To investigate if LEF-1 downregulation was specific to the granulocytic lineage, LEF-1 expression in CD14+ monocytes and CD3+ T-lymphocytes was tested. In these cell populations, LEF-1 expression levels in SCN patients were nearly identical to healthy individuals (FIGS. 5c,d). This pointed to the lineage-specific reduction of LEF-1 mRNA and protein in SCN granulocyte precursors.

To analyze if the absence of LEF-1 is a distinct feature of SCN and not common for other types of neutropenia, CD33+ cells of patients with cyclic neutropenia (CyN), idiopathic neutropenia (IN) and neutropenias associated with congenital disorders of metabolism (MN) were studied. While other LEF-1 family members, TCF-3 and TCF-4, had similar mRNA expression profiles in all neutropenias, significantly decreased (P<0.05) expression levels of LEF-1, and its target genes cyclin D1, survivin, c-Myc as well as a key granulopoietic transcription factor C/EBPalpha were found only in SCN (FIG. 1c, FIG. 5e). Interestingly, expression of β-catenin, the LEF-1 binding partner in the canonical Wnt pathway, was two-fold higher in SCN patients as compared to the other groups studied.

Pharmacological doses of G-CSF (1 to 100 μg/kg/day) are clinically effective in overcoming the “maturation arrest” of promyelocytes in SCN patients. Therefore, the expression of the aforementioned genes in SCN patients before and after G-CSF therapy was compared. Importantly, long-term G-CSF treatment had no effect on LEF-1 mRNA and protein expression in SCN patients (FIGS. 1b,c).

To investigate whether down-regulation of LEF-1 is caused by mutations in the LEF-1 promoter, sequence analysis of the known 2,700 by LEF-1 promoter was performed. No mutations was found in any of the groups studied so far, thus pointing to a regulatory defect rather than mutations in the LEF-1 promoter.

To address the significance of LEF-1 absence in CN, LEF-1 in CD34+ cells of two CN patients was re-expressed using lentiviral based constructs containing LEF-1 cDNA (LEF-1 lv). This resulted in marked up-regulation of mRNA expression of LEF-1, its target genes as well as C/EBPalpha and G-CSFR, as compared to control groups (FIG. 1d, FIGS. 6a,b). The fact that C/EBPalpha was increased by LEF-1 re-expression alone, even without G-CSF treatment, was surprising. C/EBPalpha up-regulation was not an effect of an initiated differentiation response which requires triggering by G-CSF. Intriguingly, mRNA expression levels of C/EBPalpha and G-CSFR in LEF-1 lv transduced cells were further increased in G-CSF-treated cells. Transduction of CD34+ cells with LEF-1 lv increased G-CSF-induced terminal granulocytic differentiation, while mock, or control cells remained defective in granulopoiesis, as demonstrated in FIGS. 2a,b. Indeed, LEF-1 was able to overcome the typical “maturation block” normally evident in SCN progenitors. Early increase of C/EBPalpha mRNA expression in LEF-1 rescued CD34+ SCN cells (12 and 24 hours of culture) confirmed G-CSF-independent but LEF-1-dependent regulation of C/EBPalpha (FIG. 2b, FIG. 6c). Remarkably, transduction of cells with dnLEF-1 lv, which lacks the β-catenin-binding domain, resulted in up-regulation of C/EBPalpha to a similar degree as observed with full-length LEF-1 lv (FIG. 2c).

A screen of the known 566 by upstream promoter of C/EBPalpha gene 24 revealed a putative LEF-1 binding site (−559 bp to −538 bp). LEF-1 binding in nuclear extracts from CD34+ and CD33+ cells was confirmed. In the NoShift assay, the intact consensus LEF-1 binding site of the biotinylated DNA probe was required for the binding, as shown in the competition assay with nonbiotinylated LEF-1-specific, LEF-1-nonspecific as well as mutated LEF-1-specific probes (FIG. 2d).

Specificity of the LEF-1 binding to the C/EBPa promoter is also indicated in a ChIP assay by presence of the specific band in the anti-LEF-1 precipitate and by the absence of amplicons in isotype controls (FIG. 2e). LEF-1 binds to the C/EBPalpha promoter more efficiently after induction of myeloid differentiation in CD33+ myeloid progenitors, in comparison to CD34+ cells. Together with the LEF-1-dependent C/EBPalpha expression in functional studies presented above, this data clearly indicates that LEF-1 directly regulates C/EBPalpha.

It is well known that C/EBPalpha plays a crucial role in regulating the balance between proliferation and differentiation of myeloid precursors and it is a key factor in induction of granulocyte differentiation. Targeted disruption of the C/EBPalpha gene causes a selective block in granulocytic differentiation, thus documenting the role of C/EBPalpha in this process. The data shown herein suggests that C/EBPalpha is a LEF-1 dependent differentiation factor and that LEF-1 dependent downregulation of C/EBPalpha expression in SCN patients (a pre-leukemic syndrome) leads to a maturation block in promyelocytes similar to that which has been reported for dominant-negative C/EBPalpha mutations in AML. In contrast to AML, where C/EBPalpha is mutated and therefore leads to a nonfunctional protein product, C/EBPalpha expression is down-regulated in SCN patients due to LEF-1 abrogation.

Further, the role of LEF-1 in cell proliferation and survival was investigated. LEF-1 expression in CD34+ cells from healthy individuals was inhibited using LEF-1-specific shRNA. Upon down-regulation of LEF-1 expression, we observed significant decrease of mRNA expression of its target genes (P<0.05), as compared to controls (FIG. 3a, FIG. 7a).

Moreover, a three-fold reduction in the proportion of proliferating cells after LEF-1 knockdown was found (FIG. 3c). There was no increase in the number of viable cells after LEF-1 inhibition, as 57% of proliferating cells were apoptotic (FIGS. 3d,e, FIG. 7b). This demonstrates the importance of LEF-1 not only for granulocytic differentiation, but also for the proliferation and survival of CD34+ cells. Remarkably, LEF-1 inhibition in CD34+ cells had no effect on GM-CSF-triggered differentiation towards monocytes/macrophages (FIG. 3f), suggesting a specific role for LEF-1 in the granulocytic, but probably not monocytic lineage.

In two myeloid leukemia cell lines, HL-60 and K562, LEF-1 inhibition resulted in downregulation of target genes similar to the data described above. In addition, strongly enhanced apoptosis was observed and therefore no increase in cell proliferation in both cell lines (FIGS. 8,9). Down-regulation of β-catenin in the K562 myeloid cell line did not alter their proliferation, apoptosis or LEF-1 target gene expression (FIGS. 8,9). HL-60 cells were not analyzed due to marginal β-catenin protein expression.

Observing the importance of LEF-1 for survival and proliferation of CD34+ cells, it was asked whether LEF-1 over-expression could increase their proliferation. Indeed, this led to enhanced cell proliferation, up-regulation of its target genes, including C/EBPalpha as well as G-CSFR mRNA levels (FIG. 4a-d; FIGS. 10a,b). Mock or ctrl Iv transduced CD34+ cells only gradually increased in cell number to the maximum of approximately ten-fold until day 12, followed by a subsequent decline.

Until now, reports describing the role of LEF-1 in hematopoietic cells are restricted mostly to lymphoid tissues. In accordance with the findings shown herein in myelopoiesis, others have demonstrated that LEF-1 regulates proliferation and survival of lymphoid progenitors, namely pro-, pre-B cells, and early thymic progenitors. From a broader perspective, LEF-1 is important in a particular precursor stage of lymphopoiesis and granulopoiesis (FIG. 4e). In myelopoiesis this seems to be under cytokine control, as a link between cytokine response and LEF-1 function is evident: CD34+ progenitors from two SCN patients exhibited defective granulopoiesis even in the presence of G-CSF, and this defect was corrected by reconstitution of LEF-1 expression in these cells. Physiological concentrations of G-CSF do not increase LEF-1 or C/EBPalpha expression in SCN patients (FIGS. 1b,c). However, pharmacological doses of G-CSF in vitro as we used in clinical trials (100-1,000 times over physiological)1, moderately up-regulated C/EBPalpha independently of LEF-1 (FIGS. 1d, 2c). This could explain why SCN patients respond to pharmacological doses of G-CSF in vivo.

GM-CSF has no effect on neutrophil generation in SCN patients, but only increases the number of monocytes and eosinophils. In vitro, CD34+ cells treated with LEF-1 shRNA still responded to GM-CSF with differentiation towards monocytes/macrophages but not to neutrophils; a phenotype similar to SCN in vivo. Therefore, LEF-1 is not mandatory for monopoiesis but for the granulopoiesis.

Taken together, our search for a common pathomechanism of SCN led to identification of LEF-1 as an important transcription factor regulating the differentiation of myeloid progenitors to mature neutrophils: LEF-1 is highly expressed in “healthy” promyelocytes and is abrogated in the “arrested” SCN promyelocytes. This specific phenotype could be explained by the fact that LEF-1 exerts its functions through distinct target genes. One prominent target is C/EBPalpha, which is surely a principle candidate for the mediation of differentiation: C/EBPalpha is well known to be important for neutrophil lineage-specific differentiation, and is downregulated in SCN. Additionally, LEF-1 inhibition resulted in reduced proliferation and increased apoptosis of CD34+ progenitors. These effects are most likely caused by down-regulation of cyclin D1, c-Myc and survivin; as it is observed in SCN patients. It is known that C/EBPalpha can also be anti-proliferative in certain circumstances, while we observed only a net positive effect on proliferation. It is proposed that down-regulation of cyclin D1 results in a stronger signal for growth inhibition than the effect of C/EBPalpha on proliferation. It has been found that the expression levels of cyclin D1 as well as survivin mRNA remained unchanged after C/EBPalpha inhibition, and c-Myc expression increased slightly. C/EBPalpha mediated growth arrest occurs through protein-protein interactions (e.g. p21, E2F) and is independent of DNA binding and transcriptional activity, whereas the induction of differentiation requires DNA binding. So, only the transcriptional activities of C/EBPalpha, that induce differentiation, might be directly dependent on LEF-1. Johansen et al., Mol. Cell. Biol. 21, 3789-3806, 2001 observed an inverse relation of c-Myc to C/EBPalpha which points to a context-dependent intertwined regulation of differentiation and proliferation downstream of LEF-1.

Absence of LEF-1 has a strong phenotype in CD34+ cells and in SCN, even though the expression of other TCFs (TCF-3 and TCF-4) is normal. This may be due to certain differences in their structure: only LEF-1 contains a context-dependent activating domain that interacts with ALY, TCF-3 and TCF-4 contain a CtBP-binding domain. It is known that LEF-1/TCFs genes are non redundantly required for proper mesoderm induction in Xenopus and maintenance of skin stem cells in mice. This data, in conjunction with the findings shown herein, clearly argue against redundant functions of these factors. LEF-1/TCFs may regulate different genes and may be active in different stages of proliferation and differentiation. Additionally, it has been observed that rescue of SCN progenitors with either full-length LEF-1 or dnLEF-1 resulted in up-regulation of C/EBPalpha and β-catenin inhibition caused no phenotypic differences in CD34+ cells of healthy individuals. It was also observed that neither β-catenin inhibition provoked an apparent phenotype in CD34+ progenitors (FIG. 3a-e; FIG. 7a). Therefore, it is proposed that LEF-1 regulates myelopoiesis in a β-catenin-independent manner, similar as it is known for LEF-1 regulation of T-lymphocyte development.

Since SCN is a heterogeneous syndrome and there are many SCN cases without any known gene anomalies, the definitive common pathomechanism of this syndrome is unknown. It is shown herein that in the absence of LEF-1, as found in SCN, a “maturation arrest” of myeloid progenitors occurs. Therefore, it is proposed that a common and specific molecular pathomechanism for SCN has been identified herein. Forty-six percent of the studied patients carried one of the various ELA2 mutations (Table 2), but no correlation between these mutations and LEF1 expression was observed: LEF-1 levels were hardly or not detectable irrespective of the ELA2 mutations. No mutations in G-CSFR, WASP or GFI-1 genes were detected in the group of patients investigated herein. The potential role of ELA2 in the down-regulation of LEF-1 is still unclear.

Not to be bound to theory, it is proposed that the CD34+ differentiation program towards mature neutrophils is regulated by LEF-1 through distinct mechanisms: 1) by up-regulation of proliferative and antiapoptotic genes such as cyclin D1, c-Myc and survivin, and 2) by controlling proper lineage commitment and granulocytic differentiation through regulation of C/EBPalpha (FIG. 4f).

Further experiments were conducted analysing the expression of LEF-1 in malignant blast cells from patients having T-ALL, B-ALL and AML, respectively. Different types of AML were analysed, see for further explanation table 3.

French-American-British (FAB) Classification of AML

| FAB | |
|---|---|
| subtype | Name |
| M0 | Undifferentiated acute myeloblastic |
| | leukemia |
| M1 | Acute myeloblastic leukemia with |
| | minimal maturation |
| M2 | Acute myeloblastic leukemia with |
| | maturation |
| M3 | Acute promyelocytic leukemia |
| M4 | Acute myelomonocytic leukemia |
| M4 eos | Acute myelomonocytic leukemia |
| | with eosinophilia |
| M5 | Monocytic leukemia |
| M6 | Acute erythroid leukemia |
| M7 | Acute megakaryoblastic leukemia |

According to the methods described above, RNA was isolated from the cells, reversed transcript and real-time qPTR was performed to determine the expression levels of LEF-1 mRNA in said cells. The data obtained are shown in table 4, below. Shown is the ration of LEF-1 expression versus the expression of the house-keeping gene β-actin as described above.

TABLE 4

| LEF-1 | | | AML | | |
|---|---|---|---|---|---|
| expression | AU | Mean | subtype | AU | ttest |
| T-ALL | 50.05 | 52.43 | m0 | 80.13 | 0.00001 |
| T-ALL | 12.05 | Ttest 0.0026 | m0 | 59.27 | |
| T-ALL | 31.79 | | m1 | 62.65 | 0.01 |
| T-ALL | 26.45 | | m1 | 52.21 | |
| T-ALL | 11.28 | | m1 | 25.01 | |
| T-ALL | 20.33 | | m1 | 4.92 | |
| B-ALL | 66.05 | 24.16 | m2 | 42.64 | 0.04 |
| B-ALL | 11.59 | Ttest 0.0051 | m2 | 2.80 | |
| B-ALL | 32.26 | | m2 | 5.50 | |
| B-ALL | 15.89 | | m2 | 50.71 | |
| B-ALL | 23.68 | | m4 | 2.71 | 0.11 |
| B-ALL | 16.74 | | m4 | 3.07 | |
| B-ALL | 11.12 | | m4 | 17.74 | |
| B-ALL | 25.91 | | m5 | 4.56 | 0.03 |
| B-ALL | 62.05 | | m5 | 5.01 | |
| B-ALL | 19.77 | | m6 | 3.1 | |
| B-ALL | 18.07 | | control 13 | 2.66 | |
| control 13 | 2.66 | 1.66 | control 14 | 3.85 | |
| control 14 | 3.85 | | control 4 | 0.04 | |
| control 4 | 0.04 | | control 6 | 0.81 | |
| control 6 | 0.81 | | control 8 | 1.93 | |
| control 8 | 1.93 | | control 3 | 0.69 | |
| control 3 | 0.69 | | | | |

As shown in the table 4, the ration of LEF-1/β-actin mRNA is significantly higher for ALL and various subtypes of AML in comparison to healthy controls demonstrating that determining LEF-1 is a suitable means for identifying leukaemia cells in individuals.

Next, transfection experiments transducing AML cells with LEF-1 shRNA as described above, have been performed. Briefly, blast cells from AML patients were treated with anti-LEF-1 shRNA as described above. These cells were cultured and the rate of proliferation and apoptosis were determined. As expected, transfected cells demonstrated a cell cycle arrest and the percentage of apoptotic cells was strongly increased, see FIG. 11.

Thus, using inhibitors of LEF-1 allows to direct tumor cells into cell death by inducing apoptosis. Further, proliferation of tumor cells was strongly reduced after treatment with an inhibitor of LEF-1.

### REFERENCES

1. Welte, K., Zeidler, C. & Dale, D. C. Severe congenital neutropenia. Semin. Hematol. 43, 189-195 (2006).
2. Shtutman, M. et al. The cyclin D1 gene is a target of the beta-catenin/LEF-1 pathway. Proc. Natl. Acad. Sci. USA. 96, 5522-5527 (1999).
3. He, T. C. et al. Identification of c-Myc as a target of the APC pathway. Science. 281, 1509-1512 (1998).
4. Kim, P. J., Plescia, J., Clevers, H., Fearon, E. R. & Altieri, D. C. Survivin and molecular pathogenesis of colorectal cancer. Lancet. 362, 205-209 (2003).
5. Radomska, H. S. et al. CCAAT/enhancer binding protein alpha is a regulatory switch sufficient for induction of granulocytic development from bipotential myeloid progenitors. Mol. Cell. Biol. 18, 4301-4314 (1998).
6. Zhang, P. et. al. Induction of granulocytic differentiation by 2 pathways. Blood. 99, 4406-4412, (2002).
7. Rosenberg, P. S. et al. The incidence of leukemia and mortality from sepsis in patients with severe congenital neutropenia receiving long-term G-CSF therapy. Blood. 107, 4628-4635 (2006).
8. Konishi, N. et al. Defective proliferation of primitive myeloid progenitor cells in patients with severe congenital neutropenia. Blood. 94, 4077-4083 (1999).
9. Cario, G. et al. Heterogeneous expression pattern of pro- and anti-apoptotic factors in myeloid progenitor cells of patients with severe congenital neutropenia treated with granulocyte colony-stimulating factor. Br. J. Haematol. 129, 275-278 (2005).
10. Dale, D. C. et al. Mutations in the gene encoding neutrophil elastase in congenital and cyclic neutropenia. Blood. 96, 2317-2322 (2000).
11. Germeshausen, M., Jakobs, S., Zeidler, C., & Welte, K. Update on the G-CSF receptor gene mutations in patients with severe congenital neutropenia (CN). Blood. 98, 1847 (2001).
12. Person, R. E. et al. Mutations in proto-oncogene GFI1 cause human neutropenia and target ELA2. Nat. Genet. 34, 308-312 (2003).
13. Devriendt, K. et al. Constitutively activating mutation in WASP causes X-linked severe congenital neutropenia. Nat. Genet. 27, 313-317 (2001).
14. van de Wetering, M., de Lau, W., Clevers, H. WNT signaling and lymphocyte development. Cell. 109, Suppl: 13-19 (2002).
15. Nawshad, A. & Hay, E. D. TGFbeta3 signaling activates transcription of the LEF1 gene to induce epithelial mesenchymal transformation during mouse palate development. J. Cell. Biol. 163, 1291-1301 (2003).
16. Ross, D. A. & Kadesch, T. The notch intracellular domain can function as a coactivator for LEF-1. Mol. Cell. Biol. 21, 7537-7544. (2001).
17. Merrill, B. J., Gat, U., DasGupta, R., Fuchs, E. Tcf3 and Lef1 regulate lineage differentiation of multipotent stem cells in skin. Genes and Development. 15, 1688-1705 (2001).
18. Hovanes, K. et al. β-catenin-sensitive isoforms of lymphoid enhancer factor-1 are selectively expressed in colon cancer. Nat. Genet. 28, 53-57 (2001).
19. Giese, K., Kingsley, C., Kirshner, J. R. & Grosschedl, R. Assembly and function of a TCR alpha enhancer complex is dependent on LEF-1-induced DNA bending and multiple protein-protein interactions. Genes Dev. 9, 995-1008 (1995).
20. Travis, A., Amsterdam, A., Belanger, C. & Grosschedl, R. LEF-1, a gene encoding a lymphoid-specific protein with an HMG domain, regulates T-cell receptor alpha enhancer function. Genes Dev. 5, 880-894 (1991).
21. Hsu, S.-C., Galceran, J., Grosschedl, R. Modulation of Transcriptional Regulation by LEF-1 in Response to Wnt-1 Signaling and Association with 13-Catenin. Mol. Cell. Biol. 18, 4807-4818. (1998).
22. Reya, T. et al. Wnt signaling regulates B lymphocyte proliferation through a LEF-1 dependent mechanism. Immunity. 13, 15-24 (2000).
23. Filali, M., Cheng, N., Abbott, D., Leontiev, V. & Engelhardt, J. F. Wnt-3A/beta-catenin signaling induces transcription from the LEF-1 promoter. J. Biol. Chem. 277, 33398-33410 (2002).
24. Timchenko, N. et al. Autoregulation of the Human C/EBPa Gene by Stimulation of Upstream Stimulatory Factor Binding. Mol. Cell Biol. 19, 1192-1202 (1995).
25. Suh, H. C. et al. C/EBPa determines hematopoietic cell fate in multipotential progenitor cells by inhibiting erythroid differentiation and inducing myeloid differentiation. Blood. 107, 4308-4316 (2006).
26. Ross, S. E. et al. Phosphorylation of C/EBPa Inhibits Granulopoiesis. Mol. Cell. Biol. 24, 675-686 (2004).
27. Cammenga, J. et al. Induction of C/EBPalpha activity alters gene expression and differentiation of human CD34+ cells. Blood. 101, 2206-14 (2003).
28. Zhang, D. E. et al. Absence of granulocyte colony-stimulating factor signaling and neutrophil development in CCAAT enhancer binding protein alpha-deficient mice. Proc. Natl. Acad. Sci. USA. 94, 569-574 (1997).
29. Pabst, T. et al. Dominant-negative mutations of CEBPA, encoding CCAAT/enhancer binding protein-alpha (C/EBPalpha), in acute myeloid leukemia. Nat. Genet. 27, 263-270 (2001).
30. Radomska, H. S. et al. Block of C/EBP alpha function by phosphorylation in acute myeloid leukemia with FLT3 activating mutations. J. Exp. Med. 203, 371-381 (2006).
31. Welte, K. et al. Differential effects of granulocyte-macrophage colony-stimulating factor and granulocyte colony-stimulating factor in children with severe congenital neutropenia. Blood. 75, 1056-1063 (1990).
32. Harris, T. E., Albrecht, J. H., Nakanishi, M., Darlington, G. J. CCAAT/Enhancer-binding Protein-Cooperates with p21 to Inhibit Cyclin-dependent Kinase-2 Activity and Induces Growth Arrest Independent of DNA Binding. J. Biol. Chem. 276, 29200-29209 (2001).
33. Johansen, L. M. c-Myc is a critical target for c/EBPalpha in granulopoiesis. Mol. Cell. Biol. 21, 3789-3806 (2001).
34. Waterman, M. L. Lymphoid enhancer factor/T cell factor expression in colorectal cancer. Cancer Metastasis Rev. 23, 41-52 (2004).
35. Liu, F., van den Broek, O., Destree, O., Hoppler, S. Distinct roles for Xenopus Tcf/Lef genes in mediating specific responses to Wnt/{beta}-catenin signalling in mesoderm development. Development. 132, 5375-5385 (2005).
36. Scherr, M., Battmer, K., Ganser, A. & Eder, M. Modulation of gene expression by lentiviral-mediated delivery of small interfering RNA. Cell Cycle. 2, 251-257 (2003).

TABLE 1
Oligonucleotide sequences used for NoShift
assay.

| | | Seq. ID |
|---|---|---|
| | Sequence | No. |
| WT | 5′-GTTCTGGCTTTGAAAGAGAAT-3′ | 9 |
| | 3′-ATTCTCTTTCAAAGCCAGAAC-5′ | 10 |
| Mut | 5′-GTTCTGGCTCCGAAAGAGAAT-3′ | 11 |
| | 3′-ATTCTCTTTCGGAGCCAGAAC-5′ | 12 |
| Nonspecific | 5′-AGGAGAGAAGCAAAGGACACTGC-3′ | 13 |
| competitor | 3′-GCAGTGTCCTTTGCGTCTCTCCT-5′ | 14 |

WT: LEF-1 -specific wild-type probe,
Mut: probe with a mutant LEF-1 -specific binding motif,
Nonspecific competitor: probe without LEF-1 -specific sequence.

TABLE 2
CN patients' characteristics and ELA2 mutations status

| | | Age at | ANC at | G-CSF | |
|---|---|---|---|---|---|
| | | diagnosis, | diagnosis, | treatment, | ELA2 |
| Patient | Gender | years | ×10⁹/L | μg/kg* | mutation |
| CN 1 | M | 0.1 | 0.1 | 5 | + |
| CN 2 | F | 0.5 | 0.1 | 5 | − |
| CN 3^{†} | M | 2 | 0.2 | 5 | − |
| CN 4^{†} | M | 0.3 | 0.2 | 8.3 | − |
| CN 5^{††} | F | 0.5 | 0.1 | 13 | + |
| CN 6^{††} | M | 1.3 | 0.1 | 1.2 | + |
| CN 7^{††} | M | 4.5 | 0.1 | 2.5 | + |
| CN 8 | F | 3.0 | 0.1 | 7.5 | − |
| CN 9 | M | 1.2 | 0.2 | 2.8 | − |
| CN 10 | M | 0.1 | 0 | 9 | + |
| CN 11 | F | 0.4 | 0 | 9.5 | + |
| CN 12 | F | 0.7 | 0 | 7.5 | − |
| CN 13 | F | 0.06 | 0.26 | 6.8 | − |

ANC, absolute neutrophil count
*Administration of G-CSF to all patients once per day, except for CN patients 4 and 6 (every 2^{nd} day)
^{†,††}First-grade relatives: CN 3-4, CN 5-7.

1. A use of LEF-1 or a functional fragment or homolog thereof, or an enhancer or an inducer of LEF-1 expression, activity or LEF-1 mediated signalling as an active ingredient for the preparation of a pharmaceutical for preventing or treating all types of cytopenia of the myeloid or lymphoid lineage.

2. The use according to claim 1 for preventing or treating cytopenia of the myeloid lineage.

3. The use according to claim 1 for preventing or treating neutropenia.

4. The use according to claim 3 for preventing or treating severe congenital neutropenia.

5. The use according to claim 1, wherein the active ingredient is provided in the form of a protein.

6. The use of LEF-1 according to claim 1, wherein the active ingredient is provided in form of a nucleic acid sequence.

7. The use according to claim 1, wherein the active ingredient is an inducer or enhancer of LEF-1 expression, activity or LEF-1 mediated signalling.

8. A method for preventing or treating cytopenia of the myeloid or lymphoid lineage in an individual comprising the step of administering LEF-1 or a functional fragment or homolog thereof or an inducer or enhancer of LEF-1 expression, activity, or LEF-1 mediated signalling in a therapeutically effective amount to an individual in need thereof.

9. The method according to claim 8 for preventing or treating cytopenia of the myeloid lineage.

10. The method according to claim 8 for preventing or treating neutropenia.

11. The method according to claim 8 for preventing or treating severe congenital neutropenia.

12. The method according to claim 8 further comprising the step of administering therapeutical effective amounts of G-CSF to said individual.

13. A method of treating or preventing defective myelopoiesis in an individual comprising the step of administering LEF-1 or a functional fragment or homolog thereof, or an inducer or enhancer of LEF-1 expression, activity or LEF-1 mediated signalling to individuals suffering therefrom.

14. The use of claim 1, wherein said pharmaceutical further comprises a therapeutically effective amount of G-CSF.

15. A use of an inhibitor of LEF-1 expression and/or LEF-1 mediated signalling or antagonists of LEF-1 for the preparation of a pharmaceutical for preventing or treating leukemia.

16. The use according to claim 15 for treating or preventing ALL, CML or AML.

17. The use according to claim 16 for treating or preventing AML, in particular, cancer of the myeloid lineage with transformed granulocytes.

18. The use according to claim 15, wherein the inhibitor of LEF-1 is si-RNA or shRNA, miRNA, antisense nucleic acid molecules or RNAi, small molecules or peptides acting as LEF-1-specific inhibitors.

19. The use according to claim 14, wherein the antagonist of LEF-1 is a molecule reducing or interrupting signalling via LEF-1.

20. A method of treatment or preventing cancer due to displasia of cells of the myeloid and lymphoid lineage, in particular of the AML, CML or ALL types, in an individual comprising the steps of administering a LEF-1 inhibitor or antagonist for down-regulating or inhibiting signalling via LEF-1.

21. The method according to claim 20 for the treatment of AML or CML.

22. A method for the diagnosis and/or the differentiation of different types of cytopenia comprising the step of determining the LEF-1 expression in a sample of an individual suspected to suffer from a cytopenia, in particular, suffering from neutropenia.

23. A method for the diagnosis of and/or stratification of the treatment of leukemia or cytopenia comprising the step of determining the level of LEF-1 expression in an individual.

24. The method according to claim 22, comprising the steps of a) determining the relative or absolute amount of LEF-1 or a functional fragment thereof; b) comparing the result of a) with the result determined using a control sample from a healthy individual or with an already known reference value allowing to determine the presence of absence of cytopenia or leukaemia in said individual.

25. A method for a pharmacogenetic analysis of persons undergoing treatment with a tumor therapeutic drug or undergoing pharmacogenetic studies in clinical trials comprising the step of determining the level of LEF-1 expression in said individual.

26. A method for the determination of myelodysplastic syndrome/preleukemic syndrome in an individual comprising the step of determining the expression of LEF-1 in said individual.

27. A method for mobilizing and/or inducing differentiation of stem cells comprising the step of contacting said stem cells with LEF-1 or a functional fragment or homolog thereof, or an enhancer or an inducer of LEF-1 expression, activity or LEF-1 mediated signalling.

28. Pharmaceutical composition comprising a combination of therapeutical dosages of LEF-1 and G-SCF thereof.
